(19) United States
(12) Patent Application Publication
(10) Pub. No.: US 2015/0299660 A1
(43) Pub. Date: Oct. 22, 2015

(54) DIFFERENTIATION OF HUMAN FIBROBLAST CELLS

(71) Applicant: International Stem Cell Corporation, Carlsbad, CA (US)

(72) Inventor: Trudy Christiansen-Weber, Oceanside, CA (US)

(21) Appl. No.: 14/441,083

(22) PCT Filed: Nov. 14, 2013
(86) PCT No.: PCT/US13/70167

§ 371 Date: May 6, 2015

Related U.S. Application Data
(60) Provisional application No. 61/727,025, filed on Nov. 15, 2012.

(51) Int. Cl. C12N 5/0775

(52) U.S. Cl. CPC C12N 5/0662; C12N 2506/1307

(57) ABSTRACT

The present invention is based in part on methods of differentiating fibroblast cells into adipocytes, osteocytes and chondrocytes. Additionally, the present invention provides agents and kits useful for differentiating fibroblast cells in adipocytes, osteocytes and chondrocytes. Further, the present invention provides for enhanced extracellular matrix deposition using complex sugars.

### CROSS-REFERENCE TO RELATED APPLICATIONS

This application claims the benefit of U.S. provisional application Ser. No. 61/727,025, filed Nov. 15, 2012, which is hereby incorporated by reference in its entirety, including all tables, figures, and claims

### FIELD OF THE INVENTION

The present invention relates generally to somatic stem cells, more specifically to methods for allowing human fibroblast cells to acquire a mesenchymal cell (MSC)-like state and methods of inducing the MSC-potent human fibroblasts to differentiate into adipocytes, osteocytes and chondrocytes and methods of use thereof. The present invention relates generally to the creation and enhanced deposition of extracellular matrix.

### BACKGROUND INFORMATION

Stem cells are cells found in all multicellular organisms, which can differentiate into diverse specialized cell types or self-renew to produce more stem cells. Stem cells are distinguished from other cell types by two important characteristics. First, they are unspecialized cells capable of renewing themselves through cell division, sometimes after long periods of inactivity. Second, under certain physiologic or experimental conditions, they can be induced to become tissue- or organ-specific cells with special functions. In some organs, such as the gut and bone marrow, stem cells regularly divide to repair and replace worn out or damaged tissues. In other organs, however, such as the pancreas and the heart, stem cells only divide under special conditions.

One type of stem cell is the somatic (adult) stem cell. Somatic stem cells are relatively rare undifferentiated cells found in many organs and differentiated tissues with a limited capacity for both self renewal and differentiation. Such cells vary in their differentiation capacity, but it is usually limited to cell types in the organ of origin.

Stem cells have potential in many different areas of health and medical research. Some of the most serious medical conditions, such as cancer and birth defects, are due to problems that occur when cells undergo a transformation. Understanding normal cell development and differentiation mechanisms will allow for a better understanding of these conditions.

Another potential application of stem cells is making cells and tissues for medical therapies. Today, donated organs and tissues are often used to replace those that are diseased or destroyed. Unfortunately, the number of people needing a transplant far exceeds the number of organs available for transplantation. Somatic stem cells, which would be tissue matched to the patient since the cells are derived from the patient, offer the possibility of a renewable source of replacement cells and tissues to treat a myriad of diseases, conditions, and disabilities including Parkinson's disease, amyotrophic lateral sclerosis, spinal cord injury, burns, heart disease, diabetes, and arthritis.

A fibroblast is a type of cell that synthesizes the extracellular matrix, the structural framework (stroma) for animal tissues, and plays a critical role in wound healing. The composition of the extracellular matrix determines the physical properties of the connective tissues. Fibroblasts make a variety of collagens, glycoasminoglycans, reticular and elastic fibers and glycoproteins. Fibroblasts are the most common cells of connective tissue in animals and are derived from primitive mesenchyme. Tissue damage stimulates the release of cytokines and growth factors from the extracellular matrix and induces the mitosis of fibroblasts.

Fibroblasts are commonly regarded as terminally differentiated cell type. They have limited proliferative capacity and do not give rise to other cell types. However, there is some evidence that fibroblast cells may be able to differentiate into other cell types and thus qualify as a true adult somatic stem cell. An immortal murine cell line, 3T3L1, is utilized extensively in research for its ability to differentiate into adipocytes. The cell line was derived from murine embryonic fibroblasts. In order to induce adipogenesis, standard protocol requires that the cells be plated on fibronectin, reach confluency and be treated with a combination of insulin, dexamethasone, ascorbic acid, indomethacin (COX1 inhibitor) and rosiglitazone (PPAR-gamma crosslinker). It is commonly assumed that the ability of the 3T3L1 cells to become adipocytes is a result of the cell line's embryonic origin, species and immortalization, which would confer a certain level of plasticity in phenotype. Occasionally, a random embryonic fibroblast which would harbor a few lipid droplets was observed. This observation was coupled with evidence in literature that many cell types retain a modicum of multipotetncy, such as hepatoblasts, which can be directed into more than one developmental pathway and that some cancers are believed to arise from cells which have retained an unlimited ability to proliferate while failing to differentiate. A hypothesis was developed that so-called “terminally” differentiated cells may be amenable to alteration of their cell fate if given the appropriate signals specific to the desired cell type, and thus demonstrate a previously unrecognized limited potency. It was determined that a subset of primary murine embryonic fibroblasts (roughly 20%) and chicken embryonic fibroblasts (roughly 5%), but not rat embryonic fibroblasts, could also differentiate into adipocytes if treated in a similar manner as the 3T3L1 immortal cell line. Human fibroblasts, however, while being of a similar cell type as the 3T3L1 cells, are of a different species, from a different tissue origin and are not immortal or embryonic and therefore would not necessarily conform to the expectation that a human fibroblast can become an adipocyte. Therefore, methods to induce human fibroblast cells to acquire an MSC-like state and the differentiation of these cells into adipocytes, osteocytes and chondrocytes is needed.

Extracellular matrix is the material secreted by cells which provides tensile strength, compression resistance, three dimensional organizational cues and a place of storage for growth factors and cytokines Examples of extracellular matrix at the gross anatomical level are healing wounds, tendon, ligament, bone, cartilage, blood vessels, cornea, teeth, hair and skin. The subcomponents of the extracellular matrix are varieties of keratins, collagens, elastin, nestin, calcium aptite, dentin, vitronectin, fibronectins, laminins, proteoglycans, basal membrane and less defined connective tissues.

Proteoglycans serve multiple functions. They allow water retention within tissues, bind growth factors, protease inhibitors and enzymes, participate in signal transduction and help to organize the matrix itself by binding to extracellular proteins. The sugar (or carbohydrate) chains attached to proteoglycans are called glycosaminoglycans (GAGs, also termed mucopolysaccarides). The GAGs themselves are extremely bulky with respect to their protein backbone. Aggrecan, one of the most massive proteins, is composed of 90% sugar chains.

Historically, common tissue culture practices build an extracellular matrix to any during the culturing of cells. Most cells and cell lines will live on a plate for 2-5 days before being passaged to a fresh plate. The secretion of extracellular matrix by any given cell is ongoing, but is not particularly desirable under standard tissue culture conditions because the matrix itself would pose problems in creating a single cell suspension which is necessary for good tissue culture practice and even distribution of cells on a plate. The cells may then stop proliferating and become quiescent. Such an outcome is in direct opposition to the requirement of most researchers, i.e. a rapidly dividing population which may be utilized for experiments.

When extracellular matrix is desired for tissue culture, the plate may be coated with recombinant or purified protein such as collagen, vitronectin or fibronectin. Alternatively, cells may be co-cultured with another cell type which is known to lay down an extracellular matrix necessary for cell function, albeit the matrix is far less well-defined. In both cases, researchers are still looking for proliferation of cells, but not for any of the functions that developed extracellular matrix would confer.

For tissue engineering the presence of an extracellular matrix is required. Tissue engineering is a relatively new phenomenon compared to tissue culture. Unlike tissue culture, tissue engineering utilizes synthetic substances or combinations of purified matrix proteins with cells subsequently seeded onto the scaffold. Tissue engineering requires cells to build extracellular matrix in order to form gross tissues when grafted in vivo. Another example of the need for an extracellular matrix is in studying wound healing. As summarized above however, the current cell culture technology does not allow for growth of cells on an extracellular matrix.

There is a need for methods of producing an extracellular matrix upon which to grow cells, to be used in tissue engineering as well as wound healing.

### SUMMARY OF THE INVENTION

The present invention is based in part on methods of allowing fibroblast cells to acquire a mesenchymal stem cell state of potency. Additionally, the present invention provides agents and kits useful for differentiating fibroblast cells in adipocytes, osteocytes and chondrocytes. Additionally invention also provides methods for enhanced extracellular matrix deposition using specific sugars.

Accordingly, in one embodiment, the present invention provides a method for differentiating fibroblast cells into adipocyte cells including growing fibroblast cells in fibroblast cell culture media (e.g., FibroLife S2 media, Lifeline Cell Technology, Walkersville, Md.) to confluence under standard conditions; culturing the cells in a first differentiation cell media (e.g., AdipoLife Complete DifFactor 1 media) for about 4 days; culturing the cells in a second differentiation cell media (e.g., AdipoLife Complete DifFactor 2 media) cell culture media for 17 days; and confirming the presence of adipocyte cells.

In a preferred aspect, the fibroblast cells are grown in FibroLife S2 cell culture media to confluence. The fibroblast cells are then grown in AdipoLife Complete DifFactor 1 cell culture media for about 4 days. The cells are then grown in AdipoLife Complete DifFactor 2 cell culture media for 17 days. In one aspect the presence of adipocyte cells is confirmed using Oil Red O stain to detect accumulated lipid droplets, the prime characteristic of adipocytes.

In one aspect, the AdipoLife Basal cell culture media includes Dermalife media (Lifeline Cell Technology, Walkersville, Md.); L-glutamine 1-20 mM; Plasmanate 1-20%; Dexamethasone 1-20 μM; Insulin 1-100 μg/ml; Ascorbate-2-Phosphate 1-100 μg/ml; Indomethacin 5-500 μM; EGF 1-10 ng/ml; Glycine 0.1-5 mM; Alanine 0.1-5 mM; Proline 0.1-5 mM; Biotin 0.0001-0.01 mM; Riboflavin 0.0001-0.01 mM; Vitamin B12 0.0001-0.01 mM; and Lipoic Acid 0.0001-0.01 mM.

In a preferred aspect, the AdipoLife Basal cell culture media includes Dermalife media; L-glutamine 6 mM; Plasmanate 2%; Dexamethasone 5 μM; Insulin 10 μg/ml; Ascorbate-2-Phosphate 50 μg/ml; Indomethacin 50 μM; EGF 5 ng/ml; Glycine 0.67 mM; Alanine 0.28 mM; Proline 0.35 mM; Biotin 0.00041 mM; Riboflavin 0.000266 mM; Vitamin B12 0.0010004 mM; and Lipoic Acid 0.000971 mM.

In another aspect, the DifFactor 1 supplement includes L-glutamine 1-200 mM; Plasmanate 10-50%; Dexamethasone 10-1000 μM; Insulin 10-300 μg/ml; Ascorbate-2-Phosphate 10-1000 μg/ml; Indomethacin 0.1-10 mM; EGF 10-300 μg/ml; and Troglitazone 10-1000 μM. The media is combined with AdipoLife Basal media immediately before use to create AdipoLife Complete DifFactor 1 cell culture media.

In preferred aspect, the DifFactor 1 supplement includes L-glutamine 103 mM; Plasmanate 34%; Dexamethasone 86 μM; Insulin 172 μg/ml; Ascorbate-2-Phosphate 860 μg/ml; Indomethacin 860 μM; EGF 86 μg/ml; and Troglitazone 202 μM and is combined with AdipoLife Basal media immediately before use to create AdipoLife Complete DifFactor 1 cell culture media.

In a further aspect, the DifFactor 2 supplement includes L-glutamine 1-200 mM; Plasmanate 10-50%; Dexamethasone 10-1000 μM; Insulin 10-300 μg/ml; Ascorbate-2-Phosphate 10-1000 μg/ml; Indomethacin 0.1-10 mM; and EGF 10-300 μg/ml. The media is combined with AdipoLife Basal media immediately before use to create AdipoLife Complete DifFactor 2 cell culture media.

In a preferred aspect, the DifFactor 2 supplement includes L-glutamine 103 mM; Plasmanate 34%; Dexamethasone 86 μM; Insulin 172 μg/ml; Ascorbate-2-Phosphate 860 μg/ml; Indomethacin 860 μM; and EGF 86 μg/ml and is combined with AdipoLife Basal media immediately before use to create AdipoLife Complete DifFactor 2 cell culture media.

In one embodiment, the present invention provide for adipocyte cells produced by a method including growing fibroblast cells in fibroblast cell culture media (e.g., FibroLife S2 media, Lifeline Cell Technology, Walkersville, Md.) to confluence under standard conditions; culturing the cells in a first differentiation cell media (e.g., AdipoLife Complete DifFactor 1 media) for about 4 days; culturing the cells in a second differentiation cell media (e.g., AdipoLife Complete DifFactor 2 media) cell culture media for 17 days; and confirming the presence of adipocyte cells.

In a preferred aspect, the adipocyte cells are produced by a method including growing the fibroblast cells in FibroLife S2 cell culture media to confluence. The cells are then grown in AdipoLife Complete DifFactor 1 cell culture media for about 4 days. The cells are then grown in AdipoLife Complete DifFactor 2 cell culture media for 17 days. In one aspect the presence of adipocyte cells is confirmed using Oil Red O stain to detect accumulated lipid drops, the prime characteristic of adipocytes.

In another embodiment, the invention provides a method of a method of differentiating fibroblast cells into osteocyte cells including growing fibroblast cells in fibroblast cell media (FibroLife S2 media, Lifeline Cell Technology, Walkersville, Md.) to confluence under standard conditions; culturing the cells in osteogenesis cell media (OsteoLife Complete media) for 3 weeks; and confirming the presence of osteocyte cells.

In a preferred embodiment, fibroblast cells are grown in FibroLife cell culture media to confluence. The fibroblast cells are then grown in OsteoLife Complete cell culture media for 3 weeks. In one aspect the presence of osteocyte cells is confirmed using Alizarin Red stain to detect calcium deposition, the prime characteristic of osteocytes generating bone.

In one aspect, the OsteoLife Complete cell culture media includes DMEM; L-Ala-L-Gln 1-100 mM; FBS 1-50%; EGF 0.1-20 ng/ml; bFGF 0.1-20 ng/ml; aFGF 0.1-20 ng/ml; β-Glycerophosphate 1-100 mM; Ascobate-2-Phosphate 10-1000 μg/ml; Dexamethasone 0.1-100 μM; Hyaluronic Acid 1-100 μg/ml, Glucosamine Sulfate 1-100 μg/ml and Galactose 0.1-50 g/l.

In a preferred aspect, the OsteoLife Complete cell culture media includes DMEM; L-Ala-L-Gln 6 mM; FBS 1%; EGF 5 ng/ml; bFGF 5 ng/ml; aFGF 5 ng/ml; β-Glycerophosphate 10 mM; Ascobate-2-Phosphate 50 μg/ml; Dexamethasone 0.1 μM; Hyaluronic Acid 1-100 μg/ml, Glucosamine Sulfatel-100 μg/ml and Galactose 0.1-50 g/l and confirming the present of osteocytes cells by stating with Alizarin Red stain to detect calcium deposition, the prime characteristic of osteocytes generating bone.

In another embodiment, the invention provides osteocyte cells produced by a method including growing fibroblast cells in fibroblast cell media (FibroLife S2 media, Lifeline Cell Technology, Walkersville, Md.) to confluence under standard conditions; culturing the cells in osteogenesis cell media (OsteoLife Complete media) for 3 weeks; and confirming the presence of osteocyte cells.

In a preferred embodiment, osteocyte cells are produced by a method including growing fibroblast cells in FibroLife cell culture media to confluence. The fibroblast cells are then grown in OsteoLife Complete cell culture media for 3 weeks. In one aspect the presence of osteocyte cells is confirmed using Alizarin Red stain to detect calcium deposition, the prime characteristic of osteocytes generating bone.

In another embodiment, the present invention provides a method of differentiating fibroblast cells into chondrocyte cells including growing fibroblast cells in fibroblast cell media (FibroLife S2 media, Lifeline Cell Technology, Walkersville, Md.) to 80-90% confluence under standard conditions; pelleting the cells; resuspending the cells in 1.5% alginate solution; adding alginate cell solution to 100 mM calcium chloride using a syringe to form microbeads; growing cells inside microbeads in chondrogenesis cell culture media (ChondroLife media, Lifeline Cell Technology, Walkersville, Md.) media for 3 weeks; and confirming the presence of chondrocyte cells.

In a preferred embodiment, fibroblast cells are grown in FibroLife S2 cell culture media to 80-90% confluence. The cells are then pelleted and resuspended in 1.5% alginate solution. The alginate-cell solution is then added to 100 mM calcium chloride using a syringe to form microbeads. The cells are then grown inside microbeads in ChondroLife cell culture media for 3 weeks. In one aspect the presence of chondrocyte cells is confirmed using Alcian Blue stain to detect sulfated proteoglycans, the prime characteristic of chondrocytes secreting cartilage.

In one aspect, the ChondroLife Chondrogenesis cell culture media includes FibroLife; Glucose 0.1-10 g/L; Plasmanate 1-25%; Glutamine 1-100 mM; Dexamethasone 0.1-100 μM; Insulin 0.1-20 μg/ml; PS-Transferrin 0.1-20 μg/ml; EGF 0.1-20 ng/ml; Ascorbate-2-Phosphate 10-1000 μg/ml; L-Proline 10-1000 μg/ml; TGFβ3 0.1-20 ng/ml; Glucuronic acid 1-100 μg/ml; Galactose 0.01-50 g/L; Glucosamine sulfate 1-100 μg/ml; and Hyaluronic acid 1-100 μg/ml.

In a preferred aspect, the ChondroLife Chondrogenesis cell culture media includes FibroLife; Glucose 4.5 g/L; Plasmanate 2%; Glutamine 6 mM; Dexamethasone 0.1 μM; Insulin 5 μg/ml; PS-Transferrin 5 μg/ml; EGF 5 ng/ml; Ascorbate-2-Phosphate 50 μg/ml; L-Proline 40 μg/ml; TGFβ3 2 ng/ml; Glucuronic acid 3.24 μg/ml; Galactose 2 g/L; Glucosamine sulfate 10 μg/ml; and Hyaluronic acid 10 μg/ml.

In another embodiment, the present invention provides for chondrocyte cells produced by a method including: growing fibroblast cells in fibroblast cell media (FibroLife S2 media, Lifeline Cell Technology, Walkersville, Md.) to 80-90% confluence under standard conditions; pelleting the cells; resuspending the cells in 1.5% alginate solution; adding the alginate-cell solution to 100 mM calcium chloride using a syringe to form microbeads; growing cells inside microbeads in chondrogenesis cell culture media (ChondroLife media, Lifeline Cell Technology, Walkersville, Md.) media for 3 weeks; and confirming the presence of chondrocyte cells.

In a preferred embodiment, chondrocyte cells are produced by a method including growing fibroblast cells in FibroLife S2 cell culture media to 80-90% confluence. The cells are then pelleted and resuspended in 1.5% alginate solution. The alginate-cell solution are then added to 100 mM calcium chloride using a syringe to form microbeads. The cells are then grown inside microbeads in ChondroLife cell culture media for 3 weeks. In one aspect the presence of chondrocyte cells is confirmed using Alcian Blue stain to detect sulfated proteoglycans, the prime characteristic of chondrocytes secreting cartilage.

In one embodiment, the present invention provides for a kit for differentiating fibroblast cells into adipocyte cells including AdipoLife Basal cell culture media; DifFactor 1 supplement; DifFactor 2 cell supplement; Oil Red O stain; and instructions for differentiating fibroblast cells into adipocyte cells.

In one embodiment, the present invention provides for a kit for differentiating fibroblast cells into osteocyte cells including OsteoLife Complete cell culture media; Alizarin Red stain; and instructions for differentiating fibroblast cells into osteocyte cells.

In a further embodiment, the present invention provides for a kit for differentiating fibroblast cells into chondrocyte cells including: 100 mM calcium chloride solution; ChondroLife Chondrogenesis cell culture media; Alcian Blue stain; and instructions for differentiating fibroblast cells into chondrocyte cells.

In one embodiment, the present invention provides a method of generating an extracellular matrix by culturing cells in the presence of at least one complex sugar. In one aspect, the complex sugar may be, but is not limited to, hyaluronic acid, mannose, sialic acid, chondroitin sulfate, galactose, glucuronic acid and glucosamine sulfate. In another aspect, the cells are adipocytes, osteocytes or chondrocytes derived from mesenchymal stem cells. In a further aspect, the cells may be, but are not limited to, adipocytes, osteocytes or chondrocytes derived from fibroblast cells. In an additional aspect the cells, may be, but are not limited to osteocytes, chondrocytes, blood vessels or wound healing cells.

### DETAILED DESCRIPTION OF THE INVENTION

The present invention is based in part on methods of differentiating fibroblast cells into adipocytes, osteocytes and chondrocytes. Additionally, the present invention provides agents and kits useful for differentiating fibroblast cells in adipocytes, osteocytes and chondrocytes.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

A fibroblast is a type of cell that synthesizes the extracellular matrix, the structural framework (stroma) for animal tissues, and plays a critical role in wound healing. The composition of the extracellular matrix determined the physical properties of connective tissues. Fibroblasts make a variety of collagens, glycosaminoglycans, reticular and elastic fibers and glycoproteins. They are the most common cells of connective tissue in animals and are derived from primitive mesenchyme. Tissue damage stimulates the release of cytokines and growth factors from the extracellular matrix and induces the mitosis of fibroblasts.

Mesenchymal stem cells, or MSCs, are multipotent stem cells that can differentiate into a variety of cell types. Cell types that MSCs have been shown to differentiate into in vitro or in vivo include osteoblasts, chondrocytes, myocytes, adipocytes, and, as described lately, beta-pancreatic islets cells. MSCs have a large capacity for self-renewal while maintaining their multipotency

Osteocytes are mononucleate cells that are responsible for bone formation; in essence, osteoblasts are specialized fibroblasts that in addition to fibroblastic products, express bone sialoprotein and osteocalcin.

Osteocytes produce a matrix of osteoid, which is composed mainly of Type I collagen. Osteoblasts are also responsible for mineralization of this matrix. Zinc, copper, calcium and sodium are some of the minerals required in this process. Bone is a dynamic tissue that is constantly being reshaped by osteoblasts in charge of production of matrix and mineral, and osteoclasts, which remodel the tissue. Osteocyte cells tend to decrease with age, affecting the balance of formation and resorption in the bone tissue

Adipocytes, also known as lipocytes or fat cells, are the cells that primarily compose adipose tissue, specialized in the synthesis of at or lipid and its storage as a source of energy. The adipocyte is important to the body in maintaining proper energy balance, mobilizing energy sources in response to hormonal stimulation, and commanding changes by signal secretions. Under the microscope, the adipocyte appears bloated with lipid. The nucleus of the cell is displaced to one side and the plasma membrane of the cell looks like a thin line surrounding the pool of fat.

Chondrocytes are the only cells found in cartilage. They produce and maintain the cartilaginous matrix, which consists mainly of collagen and proteoglycans. The organization of chondrocytes within cartilage differs depending upon the type of cartilage and where in the tissue they are found.

Accordingly, in one embodiment, the present invention provides a method for differentiating fibroblast cells into adipocyte cells including growing fibroblast cells in fibroblast cell culture media (e.g., FibroLife S2, Lifeline Cell Technology, Walkersville, Md.) to confluence under standard conditions; culturing the cells in a first differentiation cell media (e.g., AdipoLife Complete DifFactor 1) for about 4 days; culturing the cells in a second differentiation cell media (e.g., AdipoLife Complete DifFactor 2) cell culture media for 17 days; and confirming the presence of adipocyte cells.

In a preferred aspect, the fibroblast cells are grown in FibroLife S2 cell culture media (Table 1) to confluence. The cells are then grown in AdipoLife Complete DifFactor 1 cell culture media (AdipoLife Basal media (Table 3) plus DifFactor 1 supplement (Table 4)) for about 4 days. The cells are then grown in AdipoLife Complete DifFactor 2 cell culture media (AdipoLife Basal media (Table 3) plus DifFactor 2 supplement (Table 5)) for 17 days. In one aspect the presence of adipocyte cells is confirmed using Oil Red O stain to detect accumulated lipid droplets, the prime characteristic of adipocytes.

TABLE 1

| | FibroLife basal | FibroLife S2 | |
|---|---|---|---|
| | molarity | molarity | |
| Inorganic salts | | | |
| NH4VO3 | 1e−06 to 1e−10 | 1e−06 to 1e−10 | |
| (NH4)2MoO4—4H2O | 1e−06 to 1e−10 | 1e−06 to 1e−10 | |
| CaCl2—2H2O | 1e−01 to 1e−04 | 1e−01 to 1e−04 | |
| CuSO4—5H2O | 1e−06 to 1e−10 | 1e−06 to 1e−10 | |
| FeSO4—7H2O | 1e−03 to 1e−8 | 1e−03 to 1e−8 | |
| MgSO4—7H2O | 1e−01 to 1e−04 | 1e−01 to 1e−04 | |
| MnSO4—H2O | 1e−06 to 1e−10 | 1e−06 to 1e−10 | |
| NiCl2—6H2O | 1e−06 to 1e−10 | 1e−06 to 1e−10 | |
| KCl | 1e−01 to 1e−04 | 1e−01 to 1e−04 | |
| NaCl | 1e−01 to 1e−04 | 1e−01 to 1e−04 | |
| Na2HPO4—7H2O | 1e−01 to 1e−04 | 1e−01 to 1e−04 | |
| H2SeO3 | 1e−06 to 1e−10 | 1e−06 to 1e−10 | |
| NaSiO3—9H2O | 1e−03 to 1e−8 | 1e−03 to 1e−8 | |
| ZnSO4—7H2O | 1e−06 to 1e−10 | 1e−06 to 1e−10 | |
| Amino acids | | | |
| L-Alanine | 1e−03 to 1e−8 | 1e−03 to 1e−8 | |
| L-Arginine HCl | 1e−03 to 1e−8 | 1e−03 to 1e−8 | |
| L-Asparagine monohydrate | 1e−03 to 1e−8 | 1e−03 to 1e−8 | |
| L-Aspartic acid | 1e−03 to 1e−8 | 1e−03 to 1e−8 | |
| L-Cysteine HCl monohydrate | 1e−03 to 1e−8 | 1e−03 to 1e−8 | |
| Glycine | 1e−03 to 1e−8 | 1e−03 to 1e−8 | |
| L-Histidine HCl | 1e−03 to 1e−8 | 1e−03 to 1e−8 | |
| L-Glutamic acid | 1e−03 to 1e−8 | 1e−03 to 1e−8 | |
| L-Isoleucine | 1e−03 to 1e−8 | 1e−03 to 1e−8 | |
| L-Leucine | 1e−01 to 1e−04 | 1e−01 to 1e−04 | |
| L-Lysine HCl | 1e−01 to 1e−04 | 1e−01 to 1e−04 | |
| L-Methionine | 1e−03 to 1e−8 | 1e−03 to 1e−8 | |
| L-Phenylalanine | 1e−03 to 1e−8 | 1e−03 to 1e−8 | |
| L-Proline | 1e−03 to 1e−8 | 1e−03 to 1e−8 | |
| L-Serine | 1e−03 to 1e−8 | 1e−03 to 1e−8 | |
| L-Threonine | 1e−03 to 1e−8 | 1e−03 to 1e−8 | |
| L-Tryptophan | 1e−03 to 1e−8 | 1e−03 to 1e−8 | |
| L-Tyrosine | 1e−03 to 1e−8 | 1e−03 to 1e−8 | |
| L-Valine | 1e−01 to 1e−04 | 1e−01 to 1e−04 | |
| Vitamins | | | |
| D-Biotin | 1e−06 to 1e−10 | 1e−06 to 1e−10 | |
| Choline Chloride | 1e−03 to 1e−8 | 1e−03 to 1e−8 | |
| Folinic acid | 1e−06 to 1e−10 | 1e−06 to 1e−10 | |
| myo-inositol | 1e−03 to 1e−8 | 1e−03 to 1e−8 | |
| niacinamide | 1e−03 to 1e−8 | 1e−03 to 1e−8 | |
| D-pantothenic acid - ½ Ca | 1e−03 to 1e−8 | 1e−03 to 1e−8 | |
| Pyroxidine-HCl | 1e−03 to 1e−8 | 1e−03 to 1e−8 | |
| Riboflavin | 1e−06 to 1e−10 | 1e−06 to 1e−10 | |
| Thiamine-HCl | 1e−03 to 1e−8 | 1e−03 to 1e−8 | |
| Thioctic acid | 1e−06 to 1e−10 | 1e−06 to 1e−10 | |
| Vitamin B12 | 1e−06 to 1e−10 | 1e−06 to 1e−10 | |
| Sugars | | | |
| D-Glucose | 1e−01 to 1e−04 | 1e−01 to 1e−04 | |
| Other | | | |
| Adenine-HCl | 1e−03 to 1e−8 | 1e−03 to 1e−8 | |
| HEPES | 1e−01 to 1e−04 | 1e−01 to 1e−04 | |
| Putrescine-2HCl | 1e−06 to 1e−10 | 1e−06 to 1e−10 | |
| Pyruvic acid-Na | 1e−01 to 1e−04 | 1e−01 to 1e−04 | |
| Thymidine | 1e−06 to 1e−10 | 1e−06 to 1e−10 | |
| NaHCO3 | 1e−01 to 1e−04 | 1e−01 to 1e−04 | |
| FGF-basic | | 1-20 | ng/mL |
| Insulin | | 1-20 | μg/mL |
| L-Glutamine | | 1-100 | mM |
| Hydrocortisone hemisuccinate | | 0.1-10 | μg/mL |
| Ascorbate-2-phosphate | | 10-1000 | μg/mL |

| | |
|---|---|
| Fetal Bovine Serum | 0.5-20% |

TABLE 2
DermaLife

| | molarity |
|---|---|
| Inorganic salts | |
| NH4VO3 | 1e−06 to 1e−10 |
| (NH4)2MoO4—4H2O | 1e−06 to 1e−10 |
| CaCl2—2H2O | 1e−03 to 1e−8 |
| CuSO4—5H2O | 1e−06 to 1e−10 |
| FeSO4—7H2O | 1e−06 to 1e−10 |
| MgCl2—6H2O | 1e−03 to 1e−8 |
| MnSO4—H2O | 1e−06 to 1e−10 |
| NiCl2—6H2O | 1e−06 to 1e−10 |
| KCl | 1e−01 to 1e−04 |
| NaCl | 1e−01 to 1e−04 |
| Na2HPO4—7H2O | 1e−01 to 1e−04 |
| H2SeO3 | 1e−06 to 1e−10 |
| NaSiO3—9H2O | 1e−06 to 1e−10 |
| SnCl2—2H2O | 1e−06 to 1e−10 |
| ZnSO4—7H2O | 1e−06 to 1e−10 |
| Amino acids | |
| L-Alanine | 1e−03 to 1e−8 |
| L-Arginine HCl | 1e−03 to 1e−8 |
| L-Asparagine monohydrate | 1e−03 to 1e−8 |
| L-Aspartic acid | 1e−03 to 1e−8 |
| L-Cysteine HCl monohydrate | 1e−03 to 1e−8 |
| Glycine | 1e−03 to 1e−8 |
| L-Histidine HCl | 1e−03 to 1e−8 |
| L-Glutamic acid | 1e−03 to 1e−8 |
| L-Isoleucine | 1e−03 to 1e−8 |
| L-Leucine | 1e−03 to 1e−8 |
| L-Lysine HCl | 1e−03 to 1e−8 |
| L-Methionine | 1e−03 to 1e−8 |
| L-Phenylalanine | 1e−03 to 1e−8 |
| L-Proline | 1e−03 to 1e−8 |
| L-Serine | 1e−03 to 1e−8 |
| L-Threonine | 1e−03 to 1e−8 |
| L-Tryptophan | 1e−03 to 1e−8 |
| L-Tyrosine | 1e−03 to 1e−8 |
| L-Valine | 1e−03 to 1e−8 |
| Vitamins | |
| D-Biotin | 1e−06 to 1e−10 |
| Choline Chloride | 1e−03 to 1e−8 |
| Folinic acid | 1e−06 to 1e−10 |
| myo-inositol | 1e−03 to 1e−8 |
| niacinamide | 1e−06 to 1e−10 |
| D-pantothenic acid - ½ Ca | 1e−03 to 1e−8 |
| Pyroxidine-HCl | 1e−06 to 1e−10 |
| Riboflavin | 1e−06 to 1e−10 |
| Thiamine-HCl | 1e−03 to 1e−8 |
| Thioctic acid | 1e−03 to 1e−8 |
| Vitamin B12 | 1e−06 to 1e−10 |
| Sugars | |
| D-Glucose | 1e−01 to 1e−04 |
| Other | |
| Adenine-HCl | 1e−03 to 1e−8 |
| HEPES | 1e−01 to 1e−04 |
| Putrescine-2HCl | 1e−03 to 1e−8 |
| Pyruvic acid-Na | 1e−03 to 1e−8 |
| Sodium Acetate-3H2O | 1e−03 to 1e−8 |
| Thymidine | 1e−03 to 1e−8 |
| NaHCO3 | 1e−01 to 1e−04 |
| ethanolamine | 1e−03 to 1e−8 |
| phosphorylethanolamine | 1e−03 to 1e−8 |

TABLE 3
AdipoLife Basal
Cell Media

| Component | Final Concentration | | Preferred Concentration | |
|---|---|---|---|---|
| DermaLife | n/a | | n/a | |
| L-Glutamine | 1-20 | mM | 6 | nM |
| Plasmanate | 1-20% | | 2% | |
| Dexamethasone | 1-20 | μM | 5 | μM |
| Insulin | 1-100 | μg/mL | 10 | μg/mL |
| Ascorbate-2-Phosphate | 1-100 | μg/mL | 50 | μg/mL |
| Indomethacin | 5-500 | μM | 50 | μM |
| EGF | 1-10 | ng/mL | 5 | ng/mL |
| Glycine | 0.1-5 | mM | 0.67 | mM |
| Alanine | 0.1-5 | mM | 0.28 | mM |
| Proline | 0.1-5 | mM | 0.35 | mM |
| Biotin | 0.0001-0.01 | mM | 0.00041 | mM |
| Riboflavin | 0.0001-0.01 | mM | 0.000266 | mM |
| Vitamin B12 | 0.00-1-0.01 | mM | 0.0010004 | mM |
| Lipoic Acid | 0.0001-0.01 | mM | 0.000971 | mM |

TABLE 4
DifFactor 1
Supplement

| Component | Final Concentration | | Preferred Concentration | |
|---|---|---|---|---|
| L-Glutamine | 1-200 | mM | 103 | mM |
| Plasmanate | 10-50% | | 34% | |
| Dexamethasone | 10-1000 | μM | 86 | μM |
| Insulin | 10-300 | μg/mL | 172 | μg/mL |
| Ascorbate-2-Phosphate | 10-1000 | μg/mL | 860 | μg/mL |
| Indomethacin | 0.1-10 | mM | 860 | μM |
| EGF | 10-300 | μg/mL | 86 | μg/mL |
| Troglitazone | 10-1000 | μM | 202 | μM |

TABLE 5
DifFactor 2
Supplement

| Component | Final Concentration | | Preferred Concentration | |
|---|---|---|---|---|
| L-Glutamine | 1-200 | mM | 103 | mM |
| Plasmanate | 10-50% | | 34% | |
| Dexamethasone | 10-1000 | μM | 86 | μM |
| Insulin | 10-300 | μg/mL | 172 | μg/mL |
| Ascorbate-2-Phosphate | 10-1000 | μg/mL | 860 | μg/mL |
| Indomethacin | 0.1-10 | mM | 860 | μM |
| EGF | 10-300 | μg/mL | 86 | μg/mL |

The methods of the present invention use standard petri dishes. Previous disclosures have demonstrated the need to coat the standard plates with an additional coating, such as fibronectin, in order for the cells to attach. Using the media disclosed within the cells grow on standard cell culture dishes without the need for the additional coating. The ability to grow the cells without additional coating is a surprising aspect of the invention.

In one aspect, the AdipoLife Basal cell culture media includes Dermalife; L-glutamine 1-20 mM; Plasmanate 1-20%; Dexamethasone 1-20 μM; Insulin 1-100 μg/ml; Ascorbate-2-Phosphate 1-100 μg/ml; Indomethacin 5-500 μM; EGF 1-10 ng/ml; Glycine 0.1-5 mM; Alanine 0.1-5 mM; Proline 0.1-5 mM; Biotin 0.0001-0.01 mM; Riboflavin 0.0001-0.01 mM; Vitamin B12 0.0001-0.01 mM; and Lipoic Acid 0.0001-0.01 mM.

In a preferred aspect, the AdipoLife Basal cell culture media includes Dermalife; L-glutamine 6 mM; Plasmanate 2%; Dexamethasone 5 μM; Insulin 10 μg/ml; Ascorbate-2-Phosphate 50 μg/ml; Indomethacin 50 μM; EGF 5 ng/ml; Glycine 0.67 mM; Alanine 0.28 mM; Proline 0.35 mM; Biotin 0.00041 mM; Riboflavin 0.000266 mM; Vitamin B12 0.0010004 mM; and Lipoic Acid 0.000971 mM.

In another aspect, the DifFactor 1 supplement includes L-glutamine 1-200 mM; Plasmanate 10-50%; Dexamethasone 10-1000 μM; Insulin 10-300 μg/ml; Ascorbate-2-Phosphate 10-1000 μg/ml; Indomethacin 0.1-10 mM; EGF 10-300 μg/ml; and Troglitazone 10-1000 μM. The media is combined with AdipoLife Basal media immediately before use to create AdipoLife Complete DifFactor 1 cell culture media.

In preferred aspect, the DifFactor 1 cell culture media includes L-glutamine 103 mM; Plasmanate 34%; Dexamethasone 86 μM; Insulin 172 μg/ml; Ascorbate-2-Phosphate 860 μg/ml; Indomethacin 860 μM; EGF 86 μg/ml; and Troglitazone 202 μM and is combined with AdipoLife Basal media immediately before use to create AdipoLife Complete DifFactor 1 cell culture media.

In a further aspect, the DifFactor 2 supplement includes L-glutamine 1-200 mM; Plasmanate 10-50%; Dexamethasone 10-1000 μM; Insulin 10-300 μg/ml; Ascorbate-2-Phosphate 10-1000 μg/ml; Indomethacin 0.1-10 mM; and EGF 10-300 μg/ml. The media is combined with AdipoLife Basal media immediately before use to create AdipoLife Complete DifFactor 2 cell culture media.

In a preferred aspect, the DifFactor 2 cell culture media includes L-glutamine 103 mM; Plasmanate 34%; Dexamethasone 86 μM; Insulin 172 μg/ml; Ascorbate-2-Phosphate 860 μg/ml; Indomethacin 860 μM; and EGF 86 μg/ml and is combined with AdipoLife Basal media immediately before use to create AdipoLife Complete DifFactor 2 cell culture media.

Troglitazone (Rezulin, Resulin or Romozin) is an anti-diabetic and ant inflammatory drug, and a member of the drug class of the thiazolidinediones. Troglitazone is a ligand to both PPARα and—more strongly—PPARγ receptors. A different member of the TZDs is Rosiglitazone, which is far more exclusive to the PPARγ receptor than Troglitazone and was the original drug used to induce the 3T3L1 line into adipogenesis via the critical step of cross-linking the PPARγ receptor. Rosiglitazone worked in murine and chicken embryonic fibroblasts, but is no longer available for testing on human fibroblast cells. Troglitazone was selected as a possible replacement for cross linking the PPARγ receptor. It is not as specific as Rosiglitazone and its EC50 differs considerably between specie, but results to date indicate a minimum of 70% of the surface of the human cell culture is covered with lipid-accumulating cells when utilizing Troglitazone in combination with other elements. Troglitazone is a notably short-lived reagent for this application (3 months in powdered form, −20° C.). Our particular formulation, DifFactor 1, permits an extension of the shelf-life to at least two years when stored at −20° C.

In one embodiment, the present invention provide for adipocyte cells produced by a method including growing fibroblast cells in fibroblast cell culture media (e.g., FibroLife S2 media, Lifeline Cell Technology, Walkersville, Md.) to confluence under standard conditions; culturing the cells in a first differentiation cell media (e.g., AdipoLife Complete DifFactor 1 media) for about 4 days; culturing the cells in a second differentiation cell media (e.g., AdipoLife Complete DifFactor 2 media) cell culture media for 17 days; and confirming the presence of adipocyte cells.

In a preferred aspect, the adipocyte cells are produced by a method including growing fibroblast cells in FibroLife S2 cell culture media to confluence. The cells are then grown in AdipoLife Complete DifFactor 1 cell culture media for about 4 days. The cells are then grown in DifFactor 2 cell culture media for 17 days. In one aspect the presence of adipocyte cells is confirmed using Oil Red O stain to detect the accumulation of lipid droplets, the prime characteristic of adipocytes.

In another embodiment, the invention provides a method of a method of differentiating fibroblast cells into osteocyte cells including growing fibroblast cells in fibroblast cell media (FibroLife S2, Lifeline Cell Technology, Walkersville, Md.) to confluence under standard conditions; culturing the cells in osteogenesis cell media (OsteoLife Complete media) for 3 weeks; and confirming the presence of osteocyte cells.

In a preferred embodiment, fibroblast cells are grown in FibroLife S2 (Table 1) cell culture media to confluence. The fibroblast cells are then grown in OsteoLife Complete media (Table 6) cell culture media for 3 weeks. In one aspect the presence of osteocyte cells is confirmed using Alizarin Red stain to detect calcium deposition, the prime characteristic of osteocytes generating bone.

TABLE 6
OsteoLife
Complete

| Component | Final Concentration | | Preferred Concentration | |
|---|---|---|---|---|
| DMEM | n/a | | n/a | |
| L-Ala-L-Gln | 1-100 | mM | 6 | mM |
| Fetal Bovine Serum | 1-50% | | 1% | |
| EGF | 0.1-20 | ng/mL | 5 | ng/mL |
| bFGF | 0.1-20 | ng/mL | 5 | ng/mL |
| aFGF | 0.1-20 | ng/mL | 5 | ng/mL |
| β-Glycerophosphate | 1-100 | mM | 10 | mM |
| Ascorbate-2-Phosphate | 10-1000 | μg/mL | 50 | μg/mL |
| Dexamethasone | 0.1-100 | μM | 0.1 | nM |
| Hyaluronic Acid | 1-100 | μg/ml | 1-100 | μg/ml |
| Glucosamine Sulfate | 1-100 | μg/ml | 1-100 | μg/ml |
| Galactose | 0.1-50 | g/L | 0.1-50 | g/L |

In one aspect, the OsteoLife Complete cell culture media includes DMEM; L-Ala-L-Gln 1-100 mM; FBS 1-50%; EGF 0.1-20 ng/ml; bFGF 0.1-20 ng/ml; aFGF 0.1-20 ng/ml; β-Glycerophosphate 1-100 mM; Ascobate-2-Phosphate 10-1000 μg/ml; Dexamethasone 0.1-100 μM; Hyaluronic Acid 1-100 μg/ml; Glucosamine Sulfate 1-100 μg/ml and Galactose 0.1-50 g/L.

In a preferred aspect, the OsteoLife Complete cell culture media includes DMEM; L-Ala-L-Gln 6 mM; FBS 1%; EGF 5 ng/ml; bFGF 5 ng/ml; aFGF 5 ng/ml; β-Glycerophosphate 10 mM; Ascobate-2-Phosphate 50 μg/ml; Dexamethasone 0.1 μM; Hyaluronic Acid 1-100 μg/ml; Glucosamine Sulfate 1-100 μg/ml and Galactose 0.1-50 g/L.

In another embodiment, the invention provides osteocyte cells produced by a method including growing fibroblast cells in fibroblast cell media (FibroLife S2, Lifeline Cell Technology, Walkersville, Md.) to confluence under standard conditions; culturing the cells in osteogenesis cell media (OsteoLife Complete media) for 3 weeks; and confirming the presence of osteocyte cells.

In another aspect, the invention provides osteocyte cells produced by a method including growing fibroblast cells in FibroLife S2 cell culture media to confluence under standard conditions; culturing the cells in OsteoLife Complete cell culture media for 3 weeks; and confirming the presence of osteocyte cells by staining with Alizarin Red stain to detect calcium deposition, the prime characteristic of osteocytes generating bone.

In another embodiment, the present invention provides a method of differentiating fibroblast cells into chondrocyte cells including growing fibroblast cells in fibroblast cell media (FibroLife S2, Lifeline Cell Technology, Walkersville, Md.) to 80-90% confluence under standard conditions; pelleting the cells; resuspending the cells in 1.5% alginate solution; adding the alginate-cell solution using a syringe to 100 mM calcium chloride to form microbeads; growing cells inside microbeads in chondrogenesis cell culture media (ChondroLife, Lifeline Cell Technology, Walkersville, Md.) media for 3 weeks; and confirming the presence of chondrocyte cells.

In a preferred embodiment, fibroblast cells are grown in FibroLife S2 cell culture media to 80-90% confluence. The cells are then pelleted and resuspended in 1.5% alginate solution. The alginate-cell solution is then added using a syringe to 100 mM calcium chloride to form microbeads. The cells are then grown inside microbeads in ChondroLife cell culture media (Table 7) for 3 weeks. In one aspect the presence of chondrocyte cells is confirmed using Alcian Blue stain to detect sulfated proteoglycans, the prime characteristic of chondrocytes secreting cartilage.

TABLE 7
ChondroLife

| Component | Final Concentration | | Preferred Concentration | |
|---|---|---|---|---|
| FibroLife | n/a | | n/a | |
| Glucose | 0.1-10 | g/L | 4.5 | g/L |
| Plasmanate | 1-25% | | 2% | |
| Glutamine | 1-100 | mM | 6 | mM |
| Dexamethasone | 0.1-100 | μM | 0.1 | μM |
| Insulin | 0.1-20 | μg/mL | 5 | μg/mL |
| PS-Transferrin | 0.1-20 | μg/mL | 5 | μg/mL |
| EGF | 0.1-20 | ng/mL | 5 | ng/mL |
| Ascorbate-2-Phosphate | 10-1000 | μg/mL | 50 | μg/mL |
| L-Proline | 10-1000 | μg/mL | 40 | μg/mL |
| TGFβ3 | 0.1-20 | ng/mL | 2 | ng/mL |
| Glucuronic acid | 1-100 | μg/mL | 3.24 | μg/mL |
| Galactose | 0.1-50 | g/L | 2 | g/L |
| Glucosamine sulfate | 1-100 | μg/mL | 10 | μg/mL |
| Hyaluronic acid | 1-100 | μg/mL | 10 | μg/mL |

In one aspect, the ChondroLife Chondrogenesis cell culture media includes FibroLife; Glucose 0.1-10 g/L; Plasmanate 1-25%; Glutamine 1-100 mM; Dexamethasone 0.1-100 μM; Insulin 0.1-20 μg/ml; PS-Transferrin 0.1-20 μg/ml; EGF 0.1-20 ng/ml; Ascorbate-2-Phosphate 10-1000 μg/ml; L-Proline 10-1000 μg/ml; TGFβ3 0.1-20 ng/ml; Glucuronic acid 1-100 μg/ml; Galactose 0.01-50 g/L; Glucosamine sulfate 1-100 μg/ml; and Hyaluronic acid 1-100 μg/ml.

In a preferred aspect, the ChondroLife Chondrogenesis cell culture media includes FibroLife; Glucose 4.5 g/L; Plasmanate 2%; Glutamine 6 mM; Dexamethasone 0.1 μM; Insulin 5 μg/ml; PS-Transferrin 5 μg/ml; EGF 5 ng/ml; Ascorbate-2-Phosphate 50 μg/ml; L-Proline 40 μg/ml; TGFβ3 2 ng/ml; Glucuronic acid 3.24 μg/ml; Galactose 2 g/L; Glucosamine sulfate 10 μg/ml; and Hyaluronic acid 10 μg/ml.

In another embodiment, the present invention provides for chondrocyte cells produced by a method including growing fibroblast cells in fibroblast cell media (FibroLife S2 media, Lifeline Cell Technology, Walkersville, Md.) to 80-90% confluence under standard conditions; pelleting the cells; resuspending the cells in 1.5% alginate solution; adding the alginate-cell solution to 100 mM calcium chloride using a syringe to form microbeads; growing cells inside microbeads in chondrogenesis cell culture media (ChondroLife media, Lifeline Cell Technology, Walkersville, Md.) media for 3 weeks; and confirming the presence of chondrocyte cells.

In a preferred embodiment, the chondrocyte cells are produced by a method including growing fibroblast cells in FibroLife S2 cell culture media to 80-90% confluence. The cells are then pelleted and resuspended in 1.5% alginate solution. The alginate-cell solution is then added to 100 mM calcium chloride using a syringe to form microbeads. The cells are then grown inside microbeads in ChondroLife cell culture media for 3 weeks. In one aspect the presence of chondrocyte cells is confirmed using Alcian Blue stain to detect sulfated proteoglycans, the prime characteristic of chondrocytes secreting cartilage.

In one embodiment, the present invention provides for a kit for differentiating fibroblast cells into adipocyte cells including AdipoLife Basal cell culture media; DifFactor 1 supplement; DifFactor 2 supplement; Oil Red O stain; and instructions for differentiating fibroblast cells into adipocyte cells.

In one embodiment, the present invention provides for a kit for differentiating fibroblast cells into osteocyte cells including OsteoLife cell culture media; Alizarin Red stain; and instructions for differentiating fibroblast cells into osteocyte cells.

In a further embodiment, the present invention provides for a kit for differentiating fibroblast cells into chondrocyte cells including ChondroLife Chondrogenesis cell culture media; Alcian Blue stain; and instructions for differentiating fibroblast cells into chondrocyte cells.

Extracellular matrix is the material secreted by cells which provides tensile strength, compression resistance, three dimensional organizational cues and a place of storage for growth factors and cytokines Examples of extracellular matrix at the gross anatomical level are healing wounds, tendon, ligament, bone, cartilage, blood vessels, cornea, teeth, hair and skin. The subcomponents of the extracellular matrix are varieties of keratins, collagens, elastin, nestin, calcium aptite, dentin, vitronectin, fibronectins, laminins, proteoglycans, basal membrane and less defined connective tissues.

Proteoglycans are composed of a protein backbone with multiple chains of sugars attached. Their appearance is rather like a thread-like millipede with thousands of long legs. Examples of proteoglycans are mucous, testican, lumican, aggrecan, decorin, biglycan, versican, fibromodulin, syndecan, neurocan, glypican and perlecan.

The sugar (or carbohydrate) chains attached to proteoglycans are called glycosaminoglycans (GAGs, also termed mucopolysaccarides). They are long unbranched chains of sugars consisting of a repeating disaccharide unit composed of one six-carbon sugar (also termed hexose or hexuronic acid) and one six-carbon sugar containing nitrogen (termed hexosamine). The length and number of GAGs attached to proteoglycans varies greatly. They may also be sulfated or acetylated at different positions and in different quantities. Examples of GAGs are the varied species of heparin sulfate, heparan sulfate, heparin, chondroitin sulfate, keratin sulfate, chondroitin and dermatan sulfate. The only GAG which is not attached to a protein backbone is hyaluronic acid (or hyaluronan). GAGs are abundantly found in the extracellular matrix, the cell surface and covalently bound to membrane glycosylphosphatidylinositol. GAGs may also be found intracellularly in very limited amounts.

Examples of the components of carbohydrate chains may be glucose, mannose, sialic acid, galactose, fucose, glucuronic acid, uronic acid, hexauronic acid, iduronic acid, galactosamine and glucosamine. As mentioned above, the sugars be acetylated or sulfated at a variety of positions.

Proteoglycans serve multiple functions. They allow water retention within tissues, bind growth factors, protease inhibitors and enzymes, participate in signal transduction and help to organize the matrix itself by binding to extracellular proteins. The GAGs themselves are extremely bulky with respect to their protein backbone. Aggrecan, one of the most massive proteins, is composed of 90% sugar chains.

As used herein, the terms “complex sugar” and “complex sugars” includes monomeric and polymeric sugar chains. The use of sugars as a supplement enhancing extracellular matrix deposition is a unique aspect to the present invention. They can also be specific for cell type. As an example, previous studies have shown mesenchymal stem for chondrogenesis using a similar formulation as disclosed in the present invention, but without glucosamine sulfate. This particular complex sugar has been shown previously to be detrimental to matrix secretion from mesenchymal stem cells. Similarly, the presence of hyaluronic acid has been shown to be detrimental to matrix deposition from fibroblasts. In a surprising aspect the present invention discloses that the use of certain complex sugars in the media massively increases the accumulation of calcium derived from osteocytes while the use of other sugars actively suppresses the accumulation of calcium. For example, MSC which have under gone chondrogenesis require the use of galactose, glucuronic acid and hyaluronic acid which vastly augments cartilage formation and the use of glucosamine sulfate will repress matrix deposition. In the case of MSC which have undergone osteogenesis, galactose, glucosamine sulfate and hyaluronic acid permit vastly increased amounts of calcium deposition, while mannose or glucose is detrimental for matrix formation. Fibroblasts allowed to acquire a MSC like state require galactose, glucuronic acid and glucosamine sulfate for cartilage formation, while hyaluronic acid will repress it. Examples of sugars are hyaluronic acid, galactose, glucosamine sulfate, glucuronic acid, mannose, sialic acid, chondroitin sulfate and galactosamine.

In one embodiment, the present invention provides a method of generating an extracellular matrix by culturing cells in the presence of at least one complex sugar. In one aspect, the complex sugar may be, but is not limited to, hyaluronic acid, mannose, sialic acid, chondroitin sulfate, galactose, glucuronic acid and glucosamine sulfate. In another aspect, the cells are adipocytes, osteocytes or chondrocytes derived from mesenchymal stem cells. In a further aspect, the cells may be, but are not limited to, adipocytes, osteocytes or chondrocytes derived from fibroblast cells. The fibroblasts may acquire an MSC-like state prior to differentiation into adipocytes, osteocytes or chondrocyte. In an additional aspect the cells, may be, but are not limited to osteocytes, chondrocytes, blood vessels or wound healing cells.

The following examples are intended to illustrate but not limit the invention.

### Example 1

### Differentiation of Fibroblast Cells into Adipocytes Cells

A method was developed to differentiate fibroblast cells into adipocyte cells.

Fibroblast cells were grown in FibroLife S2 on standard tissue culture-treated plastic. Fibroblasts cells were plated at 20,000 cells/cm² and allowed the cells to grow to confluence (roughly 80,000 cells/cm²) in approximately 2 days.

Once the cells reached confluence the cell culture media was changed to AdipoLife Complete DifFactor 1 media. The cell culture media continued to be changed every 2 days. After 4 days the cell culture media was changed to AdipoLife Complete DifFactor 2 media for 17 days. Lipid accumulation was visible after 5 days culture in the AdipoLife Complete DifFactor 2 media.

After 3 weeks of culture the cells were stained with Oil Red O stained to assay for accumulated lipids

### Example 2

### Differentiation of Fibrocytes into Osteocytes

A method was developed to differentiate fibroblast cells into osteocyte cells.

Fibroblast cells were grown in FibroLife S2 on standard tissue culture-treated plastic. Fibroblasts cells were plated at 20,000 cells/cm² and allowed the cells to grow to confluence (roughly 80,000 cells/cm²) in approximately 2 days.

Once the cells reached confluence the cell culture media was changed to OsteoLife Complete media (Table 6). The cell culture media was changed every 3-4 days. The cells were grown in the OsteoLife Complete media for 3 weeks.

The cells were stained with Alizan Red assay to detect accumulated calcium. Seven days after growing the cells in the OsteoLife Complete media, calcium accumulation was visible (data not shown).

### Example 3

### Differentiation of Fibroblast Cells into Chondrocyte Cells

A method was developed to differentiate fibroblast cells into chondrocyte cells.

Fibroblast cells were grown in FibroLife S2 on standard tissue culture-treated plastic. Fibroblasts cells were plated at 20,000 cells/cm² and allowed the cells to grow to 80-90% confluence actively proliferating.

The cells were centrifuged and the cells resuspended at 2.5×10⁷ cells/ml in 1.5% alignate solution. The cells in the alignate solution are loaded into a syringe and applied to a stirred 100 mM calcium chloride solution to form microbeads. After 10 minutes, the calcium chloride solution was removed and the microbeads transferred to a 48 well plate. The microbeads were washed twice with 0.5 ml ChondroLife Chondrogenesis media (Table 7). The plates were incubated at 37° C., 5% CO2. The media was changed every 2-3 days for 3 weeks.

To examine the growth of the chondrocytes, the microbeads were fixed, frozen in OCT compound, cut in 5 μM sections and mounted on glass slides. The microbeads were stained with Alcian Blue to detect the presence of sulfated proteoglycans.

### Example 4

### Fibroblast Cells Grown Under Certain Conditions Exhibit Mesenchymal Stem Cell Markers

Fibroblast cells grown under the conditions described in Examples 1, 2 and 3 prior to differentiation were examined by flow cytometry for mesenchymal stem cell markers. The results are shown in Table 8. The results indicate that fibroblasts when grown under the conditions described herein, express markers typically found on mesenchymal stem cells. This indicates that growing fibroblast cells under the conditions described herein acquire the profile of a mesenchymal stem cell which is necessary in demonstrating its potency.

TABLE 8

| Mesenchymal | | | | | | |
|---|---|---|---|---|---|---|
| Stem Cell | Pre-culture | 48 hrs later | Fibroblast | Pre-culture | 48 hrs later | Qualification |
| Antibodies | % Positive | % Positive | Antibodies | % Positive | % Positive | Standards |
| CD31 | 0.63 | 0.67 | CD31 | 3.25* | 0.22 | <2% |
| CD166 | 99.39 | 97.7 | CD166 | 97.79 | 98.85 | >95% |
| CD45 | 0.58 | 0.36 | CD45 | 0.17 | 0.25 | <2% |
| CD90 | 99.91 | 99.72 | CD90 | 99.96 | 99.85 | >95% |
| CD44 | 99.82 | 99.91 | CD44 | 99.92 | 99.93 | >95% |
| CD29 | 100 | 100 | CD29 | 100 | 100 | >95% |
| CD14 | 0.24 | 0.39 | CD14 | 0.18 | 0.16 | <2% |
| CD105 | 99.51 | 99.96 | CD105 | 99.74 | 99.95 | >95% |
| Lin1 | 0.56 | 0.87 | Lin1 | 5.44* | 0.64 | |
| CD73 | 99.78 | 99.94 | CD73 | 100 | 100 | >95% |
| CD34 | 1.58 | 1.19 | CD34 | 0.78 | 0.32 | n/a |

*Freshly thawed, pre-culture fibroblasts fail our qualification parameters. However, after 48 hrs in which to recover, the fibroblasts match the mesenchymal stem cell qualification profile

Although the invention has been described with reference to the above example, it will be understood that modifications and variations are encompassed within the spirit and scope of the invention. Accordingly, the invention is limited only by the following claims.

What is claimed is:

1. A method for differentiating fibroblast cells into adipocyte cells comprising:
a) growing fibroblast cells in fibroblast cell culture media to confluence under standard conditions;
b) culturing the cells in a first differentiation cell culture media;
c) culturing the cells in a second differentiation cell culture media; and
d) confirming the presence of adipocyte cells.

2. The method of claim 1, wherein the cells are cultured in a first differentiation cell culture media for 4 days.

3. The method of claim 1, wherein the cells are cultured in a second differentiation cell culture media for 17 days.

4. The method of claim 1, wherein the fibroblast cell culture media is FibroLife S2 cell culture media.

5. The method of claim 1, wherein the first differentiation cell culture media is AdipoLife Complete DifFactor 1 cell culture media.

6. The method of claim 1, wherein the second differentiation cell culture media is AdipoLife Complete DifFactor 2 cell culture media.

7. The method of claim 5, wherein the AdipoLife Complete DifFactor 1 cell culture media comprises AdipoLife cell culture media and DifFactor 1 supplement.

8. The method of claim 7, wherein the DifFactor 1 supplement comprises:
a) L-glutamine 1-200 mM;
b) Plasmanate 10-50%;
c) Dexamethasone 10-1000 μM;
d) Insulin 10-300 μg/ml;
e) Ascorbate-2-Phosphate 10-1000 μg/ml;
f) Indomethacin 0.1-10 mM;
g) EGF 10-300 μg/ml; and
h) Troglitazone 10-1000 μM.

9. The method of claim 6, wherein the AdipoLife Complete DifFactor 2 cell culture media comprises AdipoLife cell culture media and DifFactor 2 supplement.

10. The method of claim 1, wherein the DifFactor 2 supplement comprises:
a) L-glutamine 1-100 mM;
b) Plasmanate 10-50%;
c) Dexamethasone 10-1000 μM;
d) Insulin 10-300 μg/ml;
e) Ascorbate-2-Phosphate 10-1000 μg/ml;
f) Indomethacin 0.1-10 mM; and
g) EGF 10-300 μg/ml.

11. The adipocyte cells produced by the method of claim 1.

12. A method of differentiating fibroblast cells into osteocyte cells comprising:
a) growing fibroblast cells in fibroblast cell culture media to confluence under standard conditions;
b) culturing the cells in osteogenesis cell culture media; and
c) confirming the presence of osteocyte cells.

13. The method of claim 12, wherein the cells are cultured in osteogenesis cell culture media for 3 weeks.

14. The method of claim 12, wherein the fibroblast cell culture media is Fibroblast S2 cell culture media.

15. The method of claim 12, wherein the osteogenesis cell culture media is OsteoLife Complete cell culture media.

16. The method of claim 15, wherein the OsteoLife Complete cell culture media comprises:
a) DMEM;
b) L-Ala-L-Gln 1-100 mM;
c) FBS 1-50%;
d) EGF 0.1-20 ng/ml;
e) bFGF 0.1-20 ng/ml;
f) aFGF 0.1-20 ng/ml;
g) β-Glycerophosphate 1-100 mM;
h) Ascobate-2-Phosphate 10-1000 μg/ml;
i) Dexamethasone 0.1-100 μM
j) Glucosamine Sulfate 1-100 μg/ml;
k) Hyaluronic Acid 1-100 μg/ml; and
l) Galactose 0.1-50 g/L.

17. The osteocyte cells produced by the method of claim 12.

18. A method of differentiating fibroblast cells into chondrocyte cells comprising:
a) growing fibroblast cells in fibroblast cell culture media to 80-90% confluence under standard conditions;
b) pelleting the cells;
c) resuspending the cells in 1.5% alginate solution;
d) adding the cells in alginate solution to 100 mM calcium chloride to form microbeads;
e) growing cells on microbeads in chondrogenesis cell culture media; and
f) confirming the presence of chondrocyte cells.

19. The method of claim 18, wherein the cells are cultured in chondrogenesis cell culture media for 3 weeks.

20. The method of claim 18, wherein the fibroblast cell culture media is Fibroblast S2 cell culture media.

21. The method of claim 18, wherein the chondrogenesis cell culture media is ChondroLife Chondrogenesis cell culture media.

22. The method of claim 21, wherein the ChondroLife Chondrogenesis cell culture media comprises:
a) FibroLife;
b) Glucose 0.1-10 g/L;
c) Plasmanate 1-25%;
d) Glutamine 1-100 mM;
e) Dexamethasone 0.1-100 μM;
f) Insulin 0.1-20 μg/ml;
g) PS-Transferrin 0.1-20 μg/ml;
h) EGF 0.1-20 ng/ml
i) Ascorbate-2-Phosphate 10-1000 μg/ml;
j) L-Proline 10-1000 μg/ml;
k) TGFβ3 0.1-20 ng/ml;
l) Glucuronic acid 1-100 μg/ml;
m) Galactose 0.1-50 g/L;
n) Glucosamine sulfate 1-100 μg/ml; and
o) Hyaluronic acid 1-100 μg/ml.

23. The chondrocyte cells produced by the method of claim 18.

24. A kit for differentiating fibroblast cells into adipocyte cells comprising:
a) AdipoLife Basal cell culture media;
b) DifFactor 1 supplement;
c) DifFactor 2 supplement;
d) Oil Red O stain; and
e) instructions for differentiating fibroblast cells into adipocyte cells.

25. A kit for differentiating fibroblast cells into osteocyte cells comprising:
a) OsteoLife cell culture media;
b) Alizarin Red stain; and
c) instructions for differentiating fibroblast cells into osteocyte cells.

26. A kit for differentiating fibroblast cells into chondrocyte cells comprising:
a) ChondroLife Chondrogenesis cell culture media;
b) Alcian Blue stain; and
c) instructions for differentiating fibroblast cells into chondrocyte cells.

27. The method of claim 1, wherein the cells are cultured on standard tissue culture treated plastic.

28. A method of generating an extracellular matrix comprising culturing cells in the presence of at least one complex sugar.

29. The method of claim 28, wherein complex sugar is selected from the group consisting of hyaluronic acid, mannose, sialic acid, chondroitin sulfate, galactose, glucuronic acid and glucosamine sulfate.

30. The method of claim 28, wherein the cells are adipocytes, osteocytes or chondrocytes derived from mesenchymal stem cells.

31. The method of claim 28, wherein the cells are adipocytes, osteocytes or chondrocytes derived from fibroblast cells.
